(19) United States
(12) Patent Application Publication
(10) Pub. No.: US 2025/0218571 A1
(43) Pub. Date: Jul. 3, 2025

(54) METHOD OF DISPLAYING RETRIEVED MEDICAL IMAGE ACCORDING TO VARIABLE RELEVANCE CRITERIA LEVEL

(71) Applicant: IRM INC., Seoul (KR)

(72) Inventor: Seung Wook CHOI, Seongnam-si (KR)

(73) Assignee: IRM INC., Seoul (KR)

(21) Appl. No.: 18/952,479

(22) Filed: Nov. 19, 2024

(30) Foreign Application Priority Data
Dec. 28, 2023 (KR) 10-2023-0193846
Feb. 28, 2024 (KR) 10-2024-0028446

(51) Int. Cl. G16H 30/20; G06F 3/04855; G06T 7/00; G16H 30/40; G16H 50/70

(52) U.S. Cl. CPC G16H 30/20; G06F 3/04855; G06T 7/0014; G16H 30/40; G16H 50/70; G06T 2207/20081; G06T 2207/20084; G06T 2207/20092

(57) ABSTRACT

A method of displaying a retrieved medical image according to an embodiment of the present disclosure may include extracting metadata of a target medical image, obtaining at least one category for the extracted metadata and a category value corresponding to each category, and displaying medical images associated with a category value determined according to a variable relevance criterion level.

### CROSS-REFERENCE TO RELATED APPLICATION

This application claims priority to and the benefit of Korean Patent Application No. 10-2023-0193846, filed on Dec. 28, 2023, No. 10-2024-0028446, filed on Feb. 28, 2024 the disclosure of which is incorporated herein by reference in its entirety.

### BACKGROUND

### 1. Field of the Invention

The present disclosure relates to a method of displaying a retrieved medical image, and more specifically, to a method of displaying a retrieved medical image according to a variable relevance criterion level.

### 2. Discussion of Related Art

One of the main tasks performed in pathology or a pathology department is to perform diagnosis by reading a patient's biological image (e.g., a patient's biological tissue slide) to determine the condition or signs of a particular disease. This type of diagnosis is a method that relies on the experience and knowledge of a skilled medical person over a long period of time. As a recent trend in diagnosis, a method of reading a slide image generated by digital imaging of the biological tissue is gradually increasing, instead of using the biological tissue slide.

Meanwhile, recently, due to the development of machine learning, attempts to automate tasks such as recognizing or classifying images by a computer system are actively being made. In particular, attempts to automate diagnoses performed by a skilled medical person using a neural network (e.g., deep learning algorithm using convolutional neural network (CNN)), which is a type of machine learning, are being made, and a representative example thereof is image-based disease diagnosis through deep learning using the neural network (e.g., CNN). In addition, auxiliary means, such as methods of retrieving images having similar features to the corresponding image when a diagnostician diagnoses a disease based on an image, may also be very useful in diagnosing a disease.

However, for a faster and more accurate diagnosis, it may be considered to provide medical histories of other patients having similar conditions to the patient for whom a medical image was taken. However, due to the rapid development of the medical field, terms defining the results obtained by analyzing medical images have not been established, making it difficult to select other medical images having similar conditions to the patient for whom the medical image was taken without a process of reading individual medical images.

Therefore, in order to more conveniently retrieve other patients having similar conditions to the patient for whom the medical image was taken, a method of automatically retrieving and displaying images similar to the captured medical image is required.

### SUMMARY OF THE INVENTION

The present disclosure is directed to providing a method of displaying a retrieved medical image.

According to an aspect of the present disclosure, there is provided a method of displaying retrieved heterogeneous types of medical images, including extracting metadata of a target medical image, assigning a category to the extracted metadata, and displaying associated medical images associated with a category determined according to a variable relevance criterion level.

In the aspect of the present disclosure, when the metadata is numerical metadata, the variable relevance criterion level may be a numerical range, and in the displaying, a medical image of the metadata included in the numerical range may be selected as the associated medical image.

In the aspect of the present disclosure, when the metadata is text metadata, the text metadata may be assigned to any one of categories hierarchized into a plurality of layers, and the displaying may include selecting any one of the plurality of layers according to the relevance criterion level, and selecting a medical image of the metadata included in the category of the selected layer as the associated medical image.

In the aspect of the present disclosure, the displaying may include setting a placement area for each assigned category, and displaying the associated medical image on each set placement area.

In the aspect of the present disclosure, the displaying may include determining the number of corresponding categories of the associated medical image and the target medical image, and placing the associated medical image in an area where the placement areas overlap when the number of categories is plural.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating an electronic device that displays a medical image according to an embodiment of the present disclosure;

FIG. 2 is a flowchart illustrating a method of displaying an associated medical image according to an embodiment;

FIG. 3 is a hierarchical diagram illustrating an embodiment for hierarchizing text metadata according to an embodiment;

FIG. 4 is a diagram illustrating a user interface element of a relevance reference level configured to be adjustable by a user according to an embodiment; and

FIG. 5 is a diagram illustrating a placement area where related medical images are displayed according to an embodiment.

### DETAILED DESCRIPTION OF EXEMPLARY EMBODIMENTS

Hereinafter, various embodiments of the present disclosure will be described in conjunction with the accompanying drawings. Various embodiments of the present disclosure may be subject to various modifications and may have various embodiments, and specific embodiments are illustrated in the drawings and described in detail. However, this is not intended to limit various embodiments of the present disclosure to specific embodiments, and it should be understood to include all modifications and/or equivalents or substitutes included in the spirit and technical scope of various embodiments of the present disclosure. In connection with the description of the drawings, similar reference numerals have been used for similar components.

In various embodiments of the present disclosure, it should be understood that the terms “includes” or “has” are intended to specify the presence of features, numbers, steps, operations, components, parts, or combinations thereof described in the specification, but do not preclude the possibility of the presence or addition of one or more other features, numbers, steps, operations, components, parts, or combinations thereof.

In various embodiments of the present disclosure, expressions such as “or” and the like include any and all combinations of the words listed together. For example, “A or B” may include A, may include B, or may include both A and B.

The expressions “first,” “second,” “primary” or “secondary” used in various embodiments of the present disclosure may modify various components of various embodiments, but do not limit the components. For example, the expressions do not limit the order and/or importance of the components, and may be used to distinguish one component from another.

When it is stated that a component is “connected” or “coupled” to another component, it should be understood that the component may be directly connected or coupled to the other component, but that a new other component may be present between the component and the other component.

In the embodiments of the present disclosure, terms such as “module,” “unit,” “part,” etc. are terms used to refer to components that perform at least one function or operation, and these components may be implemented as hardware or software, or may be implemented as a combination of hardware and software. In addition, a plurality of “modules,” “units,” “parts,” etc. may be integrated into at least one module or chip and implemented as at least one processor, except in cases where each of them needs to be implemented as a separate piece of specific hardware.

Terms such as defined in commonly used dictionaries should be interpreted as having a meaning consistent with their meaning in the context of the relevant technology, and will not be interpreted in an idealized or overly formal sense unless explicitly defined in various embodiments of the present disclosure.

Hereinafter, embodiments of the present disclosure will be described in detail with reference to the accompanying drawings.

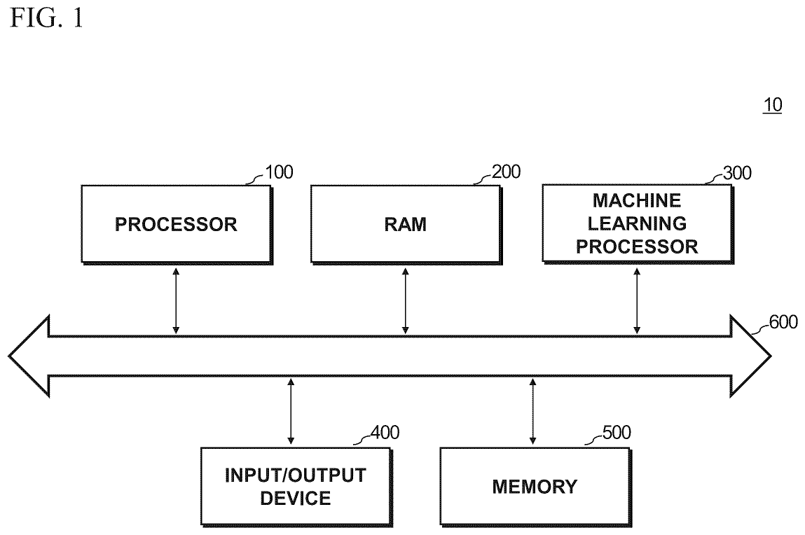

FIG. 1 is a block diagram illustrating an electronic device that displays a medical image according to an embodiment of the present disclosure.

Referring to FIG. 1, an electronic device 10 may retrieve a medical image associated with a target image, and display the retrieved medical image according to a relevance criterion level set by a user. For example, the electronic device 10 may be applied to a computing device, a personal computer, a smart TV, a smart phone, a mobile device, a video display device, a measuring device, an internet of things (IoT) device, etc., and may also be mounted on one of various types of electronic devices.

The electronic device 10 may include at least IP block and a machine learning processor 300. The electronic device 10 may include various types of IP blocks, and for example, as illustrated in FIG. 1, the electronic device 10 may include the IP blocks such as a processor 100, a random access memory (RAM) 200, an input/output device 400, and a memory 500. In addition, the electronic device 10 may further include other general-purpose components such as a multi-format codec (MFC), a video module (e.g., a camera interface, a Joint Photographic Experts Group (JPEG) processor, a video processor, a mixer, or the like), a 3D graphics core, an audio system, a display driver, a (graphics processing unit (GPU), a (digital signal processor (DSP), etc.

The components of the electronic device 10, such as the processor 100, the RAM 200, the machine learning processor 300, the input/output device 400, and the memory 500, may transmit and receive data through a system bus 600. For example, as a standard bus specification, the Advanced Microcontroller Bus Architecture (AMBA) protocol of Advanced RISC Machine (ARM) can be applied to the system bus 600, but is not limited thereto, and various types of protocols may be applied thereto.

In an embodiment, the components of the electronic device 10, such as the processor 100, the RAM 200, the machine learning processor 300, the input/output device 400, and the memory 500, may be implemented as a single semiconductor chip, and for example, the electronic device 10 may be implemented as a system on chip (SoC), but is not limited thereto, and the electronic device 10 may be implemented as a plurality of semiconductor chips. In an embodiment, the electronic device 10 may be implemented as an application processor mounted on a mobile device.

The processor 100 may control the overall operation of the electronic device 10, and as an example, the processor 100 may include at least one of a central processing unit (CPU) and a GPU. The processor 100 may include a single core or a plurality of cores (multi-core). The processor 100 may process or execute programs and/or data stored in the RAM 200 and the memory 500. For example, the processor 100 may control various functions of the electronic device 10 by executing the programs stored in the memory 500.

The RAM 200 may temporarily store programs, data, or instructions. For example, the programs and/or data stored in memory 500 may be temporarily loaded into the RAM 200 according to the control or booting code of the processor 100. The RAM 200 may be implemented using a memory such as a dynamic RAM (DRAM) or a static RAM (SRAM).

The input/output device 400 may receive input data from a user or an external source, and output a result of data processing of the electronic device 10. The input/output device 400 may be implemented using at least one of a touch screen panel, a keyboard, and various types of sensors. In an embodiment, the input/output device 400 may collect information around the electronic device 10. For example, the input/output device 400 may include at least one of various types of sensing devices such as an image-capturing device, an image sensor, a light detection and ranging (LiDAR) sensor, an ultrasonic sensor, an infrared sensor, etc., or may receive a sensing signal from the devices. In an embodiment, the input/output device 400 may sense or receive an image signal from outside the electronic device 10, and may convert the sensed or received image signal into image data, i.e., an image frame. The input/output device 400 may store the image frame in the memory 500, or provide the image frame to the machine learning processor 300.

The memory 500 is a storage location for storing data, and may store, for example, an operating system (OS), various programs, and various types of data. The memory 500 may be a DRAM, but is not limited thereto. The memory 500 may include at least one of a volatile memory or a non-volatile memory. The non-volatile memory may include a read only memory (ROM), a programmable ROM (PROM), an electrically programmable ROM (EPROM), an electrically erasable and programmable ROM (EEPROM), a flash memory, a phase-change RAM (PRAM), a magnetic RAM (MRAM), a resistive RAM (RRAM), a ferroelectric RAM (FRAM), etc. The volatile memory may include a DRAM, a SRAM, a synchronous DRAM (SDRAM), etc. In addition, in an embodiment, the memory 150 may be implemented as a storage device such as a hard disk drive (HDD), a solid-state drive (SSD), a compact flash (CF), a secure digital (SD), a micro secure digital (Micro-SD), a mini secure digital (Mini-SD), an extreme digital (xD), a memory stick, or the like.

The machine learning processor 300 may train at least one of various types of machine learning models based on previously acquired training data, and may perform computations based on the trained model. For example, the machine learning processor 300 may perform computations based on received input data to generate an inference value as a computation result, or retrain the machine learning model.

The types of machine learning models trained and inferred by the machine learning processor 300 may include supervised learning models, unsupervised learning models, and reinforcement learning models, and various types of models may be ensembled.

In addition, the machine learning processor 300 may generate a neural network model, train a neural network (or makes a neural network to learn), perform computations based on received input data, generate an information signal based on the computation result, or retrain the neural network. The neural network may include various types of neural network models such as a convolution neural network (CNN), a region with convolution neural network (R-CNN), a region proposal network (RPN), a recurrent neural network (RNN), a stacking-based deep neural network (SDNN), a state-space dynamic neural network (S-SDNN), a deconvolution network, a deep belief network (DBN), a restricted Boltzmann machine (RBM), a fully convolutional network, a long short-term memory (LSTM) network, a classification network, etc., but is not limited thereto.

The electronic device 10 according to the embodiment of the present disclosure may perform preprocessing on input data by the processor 100. Preprocessing of input data may be a process for effectively processing collected data according to the purpose. For example, the processor 100 may perform data cleaning, data transformation, data filtering, data integration, and data reduction on medical images.

The processor 100 of the electronic device 10 according to the embodiment of the present disclosure may retrieve and display medical images associated with a target medical image according to a relevance criterion level adjusted by the user. In this case, the machine learning processor 300 may analyze the target medical image to extract metadata.

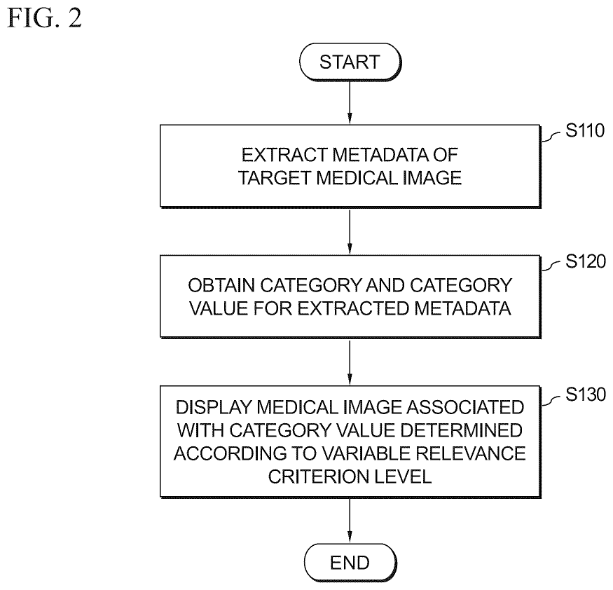

FIG. 2 is a flowchart illustrating a method of displaying an associated medical image according to an embodiment.

Referring to FIG. 2, the electronic device 10 of the present disclosure may extract metadata from the target medical image and display a medical image similar to the extracted metadata. In this case, the degree of similarity of the medical image to be displayed may be determined according to a variable relevance criterion level. The target medical image may be a medical image that a user intends to analyze, and the user may retrieve a medical image to be referenced for analysis.

In operation (S110), the electronic device 10 may extract metadata of a target medical image. The metadata is data that describes the target medical image and may be referred to as attribute information. The metadata may be divided according to a plurality of features. For example, the metadata may be divided into data related to patient personal information, data related to a size of the affected area or lesion, and data related to a location of the affected area or lesion. Metadata may be information assigned to each target medical image in order to efficiently retrieve information being sought among a large amount of information.

The metadata may be divided into numeric metadata and text metadata. For example, the numeric metadata may be patient age and/or lesion size, and the text metadata may be lesion location. However, the types of numeric metadata and text metadata of the present disclosure are not limited thereto.

According to an embodiment, the metadata may be mapped to the target medical image in advance, but the electronic device 10 may extract the metadata of the target medical image in real time based on the neural network model.

In operation (S120), the electronic device 10 may obtain a category and category value for the extracted metadata. The category may be referred to as a field identification element of the metadata, and may be an item that becomes a retrieval element when retrieving a medical image by the user. For example, categories may be divided into patient age, lesion location, and lesion size, and the category value may be a value that the metadata of the target medical image has corresponding to each category.

In operation (S130), the electronic device 10 may display a medical image associated with a category value determined according to a variable relevance criterion level. The relevance criterion level may be implemented as a user interface element so that the relevance criterion level may be independently adjusted for each category. The user may adjust the the relevance criterion level for retrieving associated medical images by adjusting the user interface element for each category.

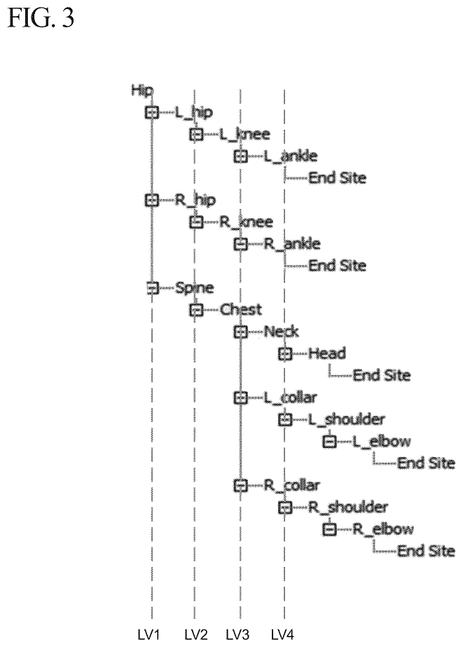

FIG. 3 is a hierarchical diagram illustrating an embodiment for hierarchizing text metadata according to an embodiment.

Referring to FIG. 3, text metadata corresponding to a medical image may be assigned to any one of a plurality of hierarchies. For example, text metadata of the affected area location may be mapped to any one of hierarchical body tissue locations. The affected area locations may be hierarchized according to locations thereof in the hierarchy based on the pelvis.

In the text metadata hierarchy of the affected area location, the left pelvis L_hip, right pelvis R_hip, and Spine may be classified as a first level LV1. The left knee L_knee, right knee R_knee, and Chest may be classified as a second level LV2. The left ankle L_ankle, right ankle R_ankle, Neck, left collarbone L_collar, and right collarbone R_collar may be classified as a second level LV2. The Head, left shoulder L_shoulder, and right shoulder R_shoulder may be classified as a second level LV2. In this case, the first level LV1 may be classified as the upper level of the second level LV2, the second level LV2 may be classified as the upper level of the third level LV3, and the third level LV3 may be classified as the upper level of the fourth level LV4.

When affected area location metadata of the target medical image is extracted as “left shoulder L_shoulder,” the electronic device 10 may designate the first level LV1 of the corresponding meta data as the Spine, the second level LV2 thereof as the Chest, the third level LV3 thereof as the left collarbone L_collar, and the fourth level LV4 thereof as the left shoulder L_shoulder.

Although FIG. 3 illustrates an embodiment in which the affected area locations are hierarchized based on a hierarchical diagram of the skeleton, the embodiment of hierarchizing text metadata in the present disclosure is not limited thereto, and may include all embodiments that can hierarchize human tissues such as organs and neural networks as well as the skeleton.

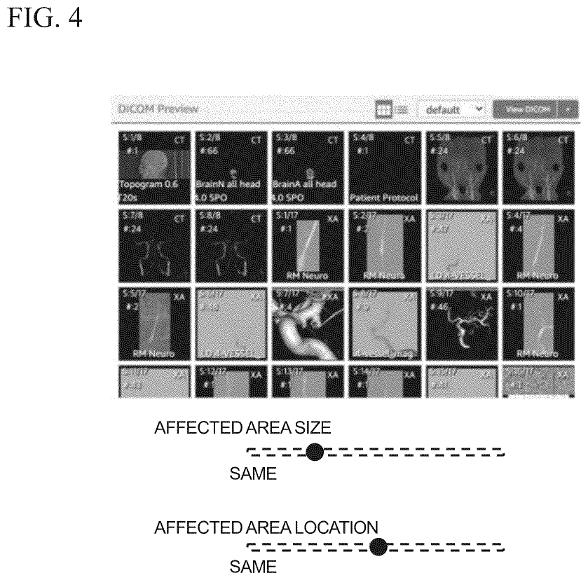

FIG. 4 is a diagram illustrating a user interface element of a relevance criterion level configured to be adjustable by a user according to an embodiment.

Referring to FIG. 4, the user interface element may be implemented to adjust the relevance criterion level, and may be implemented as a scroll element that can move horizontally or vertically, for example. The user may apply an input to move the scroll element to the electronic device 10, and the electronic device 10 may adjust the relevance criterion level in response to this.

A user interface element may be configured for each category. For example, categories may include the affected area size, the affected area location, and the patient age, and scroll elements may be implemented for each affected area size, affected area location, and patient age.

According to the embodiment of FIG. 4, when the scroll element is located at the left end, the electronic device 10 may display only the medical images whose category value of the corresponding category is the same as that of the target medical image as associated medical images.

For example, when the size of the affected area is designated as “5 cm” in the metadata and the scroll element of the affected area size category is located at the left end, the electronic device 10 may display medical images for which the metadata corresponding to the affected area size is designated as “5 cm.”

In addition, when the affected area location is designated as “left shoulder L_shoulder” in the metadata and the scroll element of the affected area location category is located at the left end, the electronic device 10 may display medical images for which the metadata corresponding to the affected area position is designated as “left shoulder L_shoulder.”

In this case, when a plurality of user interface elements capable of adjusting the relevance criterion levels are implemented, the electronic device 10 may display medical images that satisfy all of the plurality of relevance criterion levels. For example, when the affected area size of the target medical image is designated as “5 cm” in the metadata, the affected area location is designated as “left shoulder L_shoulder” in the metadata, and the scroll elements of the affected area location and affected area size categories are located at the left end, the electronic device 10 may display medical images of which the metadata corresponding to the affected area size is “5 cm” and the metadata corresponding to the affected area location is “left shoulder L_shoulder.”

When the user moves the scroll element corresponding to numerical metadata to the right, the electronic device 10 may retrieve a medical image by expanding a range of the relevance criterion level. For example, when the affected area size is designated as “5 cm” in the metadata and the scroll element of the affected area size category is moved one space from the left end, the electronic device 10 may retrieve a medical image for which the metadata corresponding to the affected area size is designated as either 4 cm or 6 cm. When the scroll element of the affected area size category is moved two spaces from the left end, the electronic device 10 may retrieve a medical image for which the metadata corresponding to the affected area size is designated as either 3 cm or 7 cm. In this way, when the scroll element corresponding to the numerical metadata is moved, the electronic device 10 may adjust the numerical range of the metadata as the relevance criterion level.

When the user moves the scroll element corresponding to the text metadata to the right, the electronic device 10 may retrieve a medical image by raising the relevance criterion level to a higher level. For example, when the affected area location is designated as “left shoulder L_shoulder” in the metadata and the scroll element of the affected area location category is moved one space from the left end, the electronic device 10 may retrieve a medical image having metadata in which the affected area location is designated as one of the lower layers of “left collarbone L_collar.” When the scroll element of the affected area location category is moved two spaces from the left end, the electronic device 10 may retrieve a medical image having metadata in which the affected area location is designated as one of the lower layers of “Chest.” That is, the electronic device 10 may retrieve a medical image of which the metadata of the affected area location has any one of “Neck,” “left collarbone L_collar,” “right collarbone R_collar,” “left shoulder L_shoulder,” and “right shoulder R_shoulder.”

That is, when the scroll element is moved one space to the right from the left end, the electronic device 10 may retrieve medical images having the same third level of the hierarchical text meta data, and when the scroll element is moved two spaces to the right from the left end, the electronic device 10 may retrieve medical images having the same second level of the hierarchical text meta data.

According to an embodiment, the electronic device 10 may set an adjustment range of the relevance criterion level corresponding to the affected area size based on the affected area location. Specifically, the electronic device 10 may set the range of the relevance reference level that is adjusted each time the user interface element corresponding to the size of the affected area moves one space based on the affected area location. For example, when the affected area location of the target medical image is “left shoulder L_shoulder,” the relevance reference level may be adjusted by ±1 cm based on the affected area size of the target medical image each time the scroll element is moved one space. In contrast, when the affected area location of the target medical image is “left ankle L_ankle,” the relevance reference level may be adjusted by ±0.5 cm based on the affected area size of the target medical image each time the scroll element is moved one space. In this way, a range of the relevance reference level to be adjusted may be pre-designated for each affected area location, but the electronic device 10 may also infer an adjustment range of the relevance reference level for each affected area location based on the neural network model.

In this case, when the relevance reference level of the affected area location is adjusted, the adjustment range of the relevance reference level of the affected area size may be changed. The adjustment range of the relevance criterion level may be the extent to which the numerical range changes per level of the relevance criterion level. For example, when a medical image of an affected area location corresponding to a hierarchical affected area location at the fourth level is retrieved, the relevance reference level may be ±1 cm, but when a medical image of an affected area location corresponding to a hierarchical affected area location at the third level is retrieved, the relevance criterion level may be ±2 cm.

That is, even if the scroll element corresponding to the numerical metadata is moved one space, the electronic device 10 may set a numerical range corresponding to one space of the scroll element differently according to the relevance standard level corresponding to the text metadata. Accordingly, the electronic device 10 does not uniformly adjust the numerical range corresponding to the numerical metadata, but may set the numerical range wide when the location of the affected area corresponds to a large tissue, and may set the numerical range small when the location of the affected area corresponds to a small tissue, thereby enabling efficient retrieval appropriate for the situation.

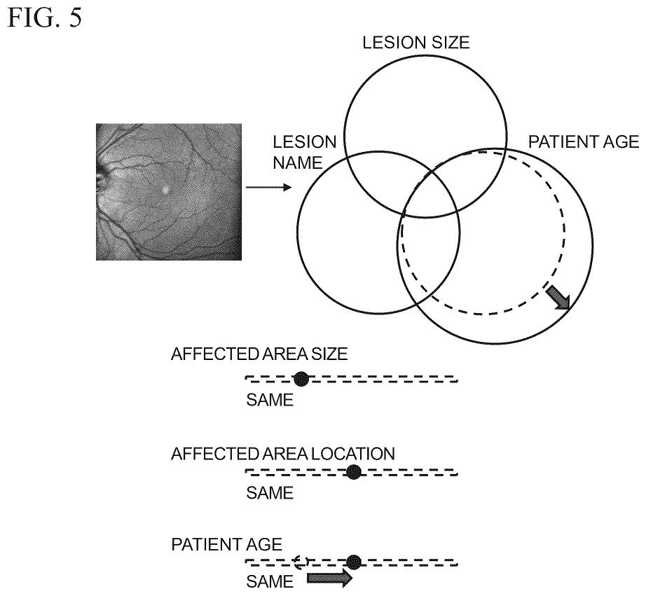

FIG. 5 is a diagram illustrating a placement area where related medical images are displayed according to an embodiment.

Referring to FIG. 5, when the metadata is divided into a plurality of categories and the scroll elements are configured to be independently adjustable for each category, the electronic device 10 may display only the medical images that satisfy all of the relevance criteria levels corresponding to each category, but may also display medical images that satisfy only one relevance criteria level.

In this case, a placement area where a medical image satisfying a first relevance criterion level corresponding to a first feature of metadata (e.g., an affected area size) is displayed may be referred to as a first placement area, a placement area where a medical image satisfying a second relevance criterion level corresponding to a second feature of metadata (e.g., an affected area location) is displayed may be referred to as a second placement area, and a placement area where a medical image satisfying a third relevance criterion level corresponding to a third feature of metadata (e.g., the patient age) is displayed may be referred to as a third placement area.

The electronic device 10 may display a medical image satisfying both the first relevance criterion level and the second relevance criterion level in a first overlapping area where the first placement area and the second placement area overlap, display a medical image satisfying both the second relevance criterion level and the third relevance criterion level in a second overlapping area where the second placement area and the third placement area overlap, and display a medical image satisfying both the first relevance criterion level and the third relevance criterion level in a third overlapping area where the first placement area and the third placement area overlap. The electronic device 10 may display a medical image satisfying all of the first relevance criterion level to the third relevance criterion level in an intersection area where the first overlapping area, the second overlapping area, and the third overlapping area all intersect.

In this case, since the medical image displayed in the intersection area, the medical image displayed in the overlapping area, and the medical image displayed in the non-overlapping placement area may have a high relevance to the target image in this order, the size of the intersection area may be larger than the size of the overlapping area, and the size of the overlapping area may be larger than the size of the non-overlapping placement area.

Alternatively, the electronic device 10 may set the sizes of medical images to be displayed differentially so that the user may preferentially identify the medical image of high importance. For example, the size of the medical image included in the intersection area may be larger than the size of the medical image included in the overlapping area, and the size of the medical image included in the overlapping area may be larger than the size of the medical image included in the non-overlapping placement area.

According to an embodiment, when the relevance criterion level is adjusted, the size of the placement area corresponding to the corresponding category may be adjusted. For example, when the range of the relevance criterion level is widened by the movement of the scroll element, the electronic device 10 may increase the size of the placement area corresponding to the corresponding category. In contrast, when the range of the relevance criterion level is narrowed by the movement of the scroll element, the electronic device 10 may reduce the size of the placement area corresponding to the corresponding category.

Meanwhile, the methods according to the various embodiments of the present invention described above may be implemented in the form of an application or software program that can be installed on an existing electronic device.

In addition, all or part of the method may be configured as several software function modules and implemented in an OS. Alternatively, each step may be configured as one software function module, or respective steps may be combined to be configured as one software function module and implemented in the OS. Therefore, even if some embodiments of the present disclosure are not implemented entirely as one software function module, when several software function modules implement each step of the present disclosure and several software function modules are implemented in one OS, it may be understood that the method of the present disclosure is implemented.

In addition, the methods according to the various embodiments of the present invention described above may be implemented only with a software upgrade or a hardware upgrade for an existing electronic device. In addition, the various embodiments of the present invention described above may also be performed through an embedded server equipped in an electronic device, or an external server of the electronic device.

Meanwhile, according to an embodiment of the present invention, the various embodiments described above may be implemented as software including instructions stored in a computer readable recording medium that can be read by a computer or a similar device using software, hardware, or a combination thereof. In some cases, the embodiments described in this specification may be implemented by the processor itself. According to a software implementation, embodiments such as the procedures and functions described in this specification may be implemented as separate software modules. Each of the software modules may perform one or more functions and operations described in this specification.

Meanwhile, a computer or a similar device is a device capable of calling a stored instruction from a storage medium and operating according to the called instruction, and may include a device according to the disclosed embodiments. When the instruction is executed by a processor, the processor may perform a function corresponding to the instruction directly or by using other components under the control of the processor. The instructions may include code generated or executed by a compiler or an interpreter.

A recording medium that can be read by a device may be provided in the form of a non-transitory computer readable recording medium. Here, “non-transitory” only means that the storage medium does not contain signals and is tangible, and does not distinguish between whether data is stored semi-permanently or temporarily in the storage medium. In this case, the non-transitory computer readable medium means a medium that stores data semi-permanently and can be read by a device, rather than a medium that stores data for a short period of time, such as a register, a cache, or a memory. Specific examples of the non-transitory computer-readable media may include CDs, DVDs, hard disks, Blu-ray discs, USBs, memory cards, ROMs, etc.

According to an embodiment of the present disclosure, medical images associated with the target image can be retrieved and displayed according to the relevance criterion level that can be adjusted by a user. Accordingly, the user can easily retrieve a desired medical image, and can display medical images preferentially according to importance, thereby enabling efficient medical image review.

The effects that can be obtained from the exemplary embodiments of the present disclosure are not limited to the effects mentioned above, and other effects that are not mentioned can be clearly derived and understood by a person having ordinary skill in the art to which the exemplary embodiments of the present disclosure belong from the following description. That is, unintended effects resulting from implementing the exemplary embodiments of the present disclosure can also be derived by a person having ordinary skill in the art from the exemplary embodiments of the present disclosure.

As described above, exemplary embodiments have been disclosed in the drawings and specifications. Although embodiments have been described using specific terms in the present specification, the terms have been used only for the purpose of describing the technical idea of the present disclosure and have not been used to limit the meaning or the scope of the present disclosure described in the claims. Therefore, those of ordinary skill in the art will understand that various modifications and other equivalent embodiments are possible from the disclosed exemplary embodiments. Therefore, the true technical protection scope of the present disclosure should be determined by the technical idea of the accompanying patent claims.

What is claimed is:

1. A method of displaying a retrieved medical image, comprising:
extracting metadata of a target medical image;
obtaining at least one category for the extracted metadata and a category value corresponding to each category; and
displaying medical images associated with a category value determined according to a variable relevance criterion level.

2. The method of claim 1,
wherein when the metadata is numerical metadata, the variable relevance criterion level is a numerical range, and
the displaying includes:
identifying a numerical range corresponding to the variable relevance criterion level; and
selecting a medical image corresponding to metadata included in the numerical range as the associated medical image.

3. The method of claim 1,
wherein when the metadata is text metadata, the text metadata is assigned to any one of categories hierarchized into a plurality of layers, and
the displaying includes:
selecting any one of the plurality of layers according to the relevance criterion level; and
selecting a medical image of metadata included in the category of the selected layer as the associated medical image.

4. The method of claim 1,
wherein the metadata is divided according to a plurality of features, and a distinct user interface element that allows the relevance criterion level to be adjusted for each divided metadata is implemented, and
the displaying includes:
setting a placement area of the associated medical image for each classified metadata; and
displaying the associated medical image on each set placement area.

5. The method of claim 4, further comprising:
displaying an associated medical image satisfying a first relevance criterion level corresponding to a first feature related to the metadata on a first placement area; and
displaying an associated medical image satisfying a second relevance criterion level corresponding to a second feature on a second placement area.

6. The method of claim 5,
wherein the setting of the placement area includes:
setting an overlapping area in which the first placement area and the second placement area overlaps partially; and
displaying an associated medical image satisfying both the first relevance criterion level and the second relevance criterion level on the overlapping area.

7. The method of claim 6, wherein a size of the overlapping area is larger than a size of a non-overlapping placement area, or a size of the associated medical image included in the overlapping area is larger than a size of an associated medical image included in the non-overlapping placement area.
